SEQUENCE LISTING (28 sequences)

SEQ ID NO: 1 - DNA, 18 bp, Artificial Sequence
  Description of Artificial Sequenceprimer CJ_2F derived from Sequence 2, primer CHX-CJ-2F
ggattccactgcactcca

SEQ ID NO: 2 - DNA, 24 bp, Artificial Sequence
  Description of Artificial Sequenceprimer CJ_2R derived from Sequence 2, primer CHX-CJ-2R
cagttgctgtgtagctatcccttt

SEQ ID NO: 3 - DNA, 20 bp, Artificial Sequence
  Description of Artificial Sequenceprimer CJ_1F derived from Sequence 1
agcctgggtgacagaatgac

SEQ ID NO: 4 - DNA, 20 bp, Artificial Sequence
  Description of Artificial Sequenceprimer CJ_1R derived from Sequence 1
tcagggcttatttccaggag

SEQ ID NO: 5 - DNA, 25 bp, Artificial Sequence
  Description of Artificial Sequenceprimer CJ_3F derived from Sequence 3
gcattgtgaactctcaagctttatt

SEQ ID NO: 6 - DNA, 20 bp, Artificial Sequence
  Description of Artificial Sequenceprimer CJ_3R derived from Sequence 3
cctgggtgacagaatgagac

SEQ ID NO: 7 - DNA, 25 bp, Artificial Sequence
  Description of Artificial Sequenceprimer CJ_5F derived from Sequence 3
tctcaagctttattttctaatctga

SEQ ID NO: 8 - DNA, 25 bp, Artificial Sequence
  Description of Artificial Sequenceprimer CJ_5R derived from Sequence 3
aactaatatatagtgaagattgacg

SEQ ID NO: 9 - DNA, 278 bp, Artificial Sequence
  Description of Artificial SequenceSequence 1 present in presumptive Creutzfeldt-Jakob Disease (CJD) derived from PCR products using primers 75F/83R
actatagaatactcaagctatgcatccaacgcgttgggagctctcccatatggtccacttgcaggcggccgcactagtgattccactgcactccagcctgggtgacagaatgacactgtttctaaaaaaacaaaacaaaacaaaacaaaaaaaattctgcatttttttataaggatctgctttaactctaactgctcctggaaataagccctgactaatcaaggctacacagcaactgtgtagaatcccgcggccatggcggccgggagcatgcgacg

SEQ ID NO: 10 - DNA, 320 bp, Artificial Sequence
  Description of Artificial SequenceSequence 2 present in presumptive Creutzfeldt-Jakob Disease (CJD) derived from PCR products using primers 75F/83R
gtaaaacgacggccagtgaattgtaatacgactcactatagggcgaattkrgccmgrcgatsgsmkgctccygkccggcsmtggccgcgggattccactgcactccagcctgggcgacagagcaagactccatctcaaaaaacaaacaaaaaacaatcatatgatccagcaatcccactactgggaattyatggaaaggaaaagaaatcagtgtatcaaagggatagctacacagcaactgtgtagaatcactagtgcggccgcctgcaggtcgaccatatgggagagctcccaacgcgttggatgcatagcttgagtat

SEQ ID NO: 11 - DNA, 312 bp, Artificial Sequence
  Description of Artificial SequenceSequence 3 present in presumptive Creutzfeldt-Jakob Disease (CJD) derived from PCR products using primers 75F/83R
ggcgagcctgtccggcgctggccgggatcacccttbtggagcattgtgaactctcaagctttattttctaatctgaaaatgagggagaacagtaactaccttataggttatgtggattaaatgagatagtgccccgtcaatcttcactatatattagttataatcattttttttttctttgagacagggtctcattctgtcacccaggctggagtgcagtggaatcactagtgcggccgcctgcaggtcgaccatatgggagagctcccaacgcgttggatgcatagcttgagtattctatagtgtcaccta

SEQ ID NO: 12 - DNA, 18 bp, Artificial Sequence
  Description of Artificial SequencePCR primer 75F from non-coding region of human prion gene
ccactgcactccagcctg

SEQ ID NO: 13 - DNA, 18 bp, Artificial Sequence
  Description of Artificial SequencePCR primer 75R from non-coding region of human prion gene
caggctggagtgcagtgg

SEQ ID NO: 14 - DNA, 21 bp, Artificial Sequence
  Description of Artificial SequencePCR primer 76R from non-coding region of human prion gene
ggtctccaggtctgttggatc

SEQ ID NO: 15 - DNA, 22 bp, Artificial Sequence
  Description of Artificial SequencePCR primer 77F from non-coding region of human prion gene
cacactgatatgccttatgcgc

SEQ ID NO: 16 - DNA, 22 bp, Artificial Sequence
  Description of Artificial SequencePCR primer 77R from non-coding region of human prion gene
ccgcataaggcatatcagtgtg

SEQ ID NO: 17 - DNA, 23 bp, Artificial Sequence
  Description of Artificial SequencePCR primer 78R from non-coding region of human prion gene
cctccactttattgagcacttag

SEQ ID NO: 18 - DNA, 22 bp, Artificial Sequence
  Description of Artificial SequencePCR primer 79F from non-coding region of human prion gene
ctcacataaacatggcccaggc

SEQ ID NO: 19 - DNA, 23 bp, Artificial Sequence
  Description of Artificial SequencePCR primer 80F from non-coding region of human prion gene
gcatctaagtgggcttagcactg

SEQ ID NO: 20 - DNA, 24 bp, Artificial Sequence
  Description of Artificial SequencePCR primer 81R from non-coding region of human prion gene
gattgaactcaattatgtttatgc

SEQ ID NO: 21 - DNA, 23 bp, Artificial Sequence
  Description of Artificial SequencePCR primer 82R from non-coding region of human prion gene
gctggtctcaaactcctcacctc

SEQ ID NO: 22 - DNA, 20 bp, Artificial Sequence
  Description of Artificial SequencePCR primer 83R from non-coding region of human prion gene
ctacacagttgctgtgtagc

SEQ ID NO: 23 - DNA, 27 bp, Artificial Sequence
  Description of Artificial SequenceSequence 1 (Alu-Sx) circulating nucleic acid (CNA) fragment
ccactgcactccagcctgggtgacaga

SEQ ID NO: 24 - DNA, 27 bp, Artificial Sequence
  Description of Artificial SequenceAlu sequence homologous to Sequence 1 (Alu-Sx) circulating nucleic acid (CNA) fragment
ccactgcactccagcctgggggacaga

SEQ ID NO: 25 - DNA, 41 bp, Artificial Sequence
  Description of Artificial SequenceSequence 2 (ALU-Sx) circulating nucleic acid (CNA) fragment
ccactgcactccagcctgggcgacagagcaagactccatct

SEQ ID NO: 26 - DNA, 41 bp, Artificial Sequence
  Description of Artificial SequenceAlu sequence homologous to Sequence 2 (ALU-Sx) circulating nucleic acid (CNA) fragment
ccactgcactccagcctgggggacagagcaagactccatct

SEQ ID NO: 27 - DNA, 27 bp, Artificial Sequence
  Description of Artificial SequenceSequence 3 (ALU-J) circulating nucleic acid (CNA) fragment
tctgtcacccaggctggagtgcagtgg

SEQ ID NO: 28 - DNA, 27 bp, Artificial Sequence
  Description of Artificial SequenceAlu sequence homologous to Sequence 3 (ALU-J) circulating nucleic acid (CNA) fragment
tctgtcccccaggctggagtgcagtgg

(19) United States
(12) Patent Application Publication
(10) Pub. No.: US 2006/0099621 A1
(43) Pub. Date: May 11, 2006

(54) Detection of nucleic acids to assess risk for Creutzfeldt-Jakob disease

(75) Inventors: Ekkehard Schuetz, Gottingen (DE); Leonid Iakoubov, Pleasanton, CA (US); Howard Urnovitz, Iowa City, IA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Chronix Biomedical, San Jose, CA (US)

(21) Appl. No.: 11/245,514

(22) Filed: Oct. 7, 2005

Related U.S. Application Data
(60) Provisional application No. 60/616,726, filed on Oct. 7, 2004.

(51) Int. Cl. C12Q 1/68; C12P 19/34

(52) U.S. Cl. 435/6; 435/91.2

(57) ABSTRACT

The present invention provides a method of detecting abnormal circulating nucleic acid profiles to assess the risk of Creutzfeldt-Jakob Disease.

### CROSS-REFERENCE TO RELATED APPLICATIONS

This application claims benefit of U.S. provisional application no. 60/616,726, filed Oct. 7, 2004, which application is herein incorporated by reference.

### BACKGROUND OF THE INVENTION

Creutzfeldt-Jakob Disease (CJD) is a very rare neurodegenerative disease that is invariably fatal. It is a member of a family of human and animal diseases known as the transmissible spongiform encephalopathies (TSEs). CJD is the most common of the known human TSEs, which include kuru, fatal familial insomnia, and Gerstmann-Straussler-Scheinker disease (GSS). Other TSEs occur in animals. These include bovine spongiform encephalopathy (BSE); scrapie, which can be found in sheep and goats; mink encephalopathy; and feline encephalopathy. Similar diseases have occurred in elk, deer, and exotic zoo animals. TSE models have also been described in experimental animals models such as mice and hamsters.

There are three forms of CJD: hereditary, sporadic, and acquired. In acquired CJD, the disease is believed to be transmitted by exposure to brain or nervous system tissue. The appearance of the new variant of CJD (nv-CJD or v-CJD) in younger patient in Great Britain and France has led to concern that BSE is transmitted to humans through consumption of contaminated beef. The risk of acquiring the various forms of CJD from blood transfusions is also becoming of increasing concern with the identification of a second case of CJD that arose from a blood transfusion from a donor who died having CJD (Peden et al., Lancet 364:527, 2004). In addition, three United Kingdom patients died having CJD where the blood donors showed no clinical signs of CJD. There is no reliable method for determining whether a patient is at risk for CJD from contaminated blood or other tissues. The current invention addresses this need.

### BRIEF SUMMARY OF THE INVENTION

This invention is based on the discovery that abnormal nucleic acid profiles are detected in acellular fluid samples, e.g., serum or plasma, from humans at risk for transmissible spongiform encephalopathy, e.g., CJD and vCJD. The invention therefore provides a method of detecting a human at increased risk for CJD, the method comprising: incubating nucleic acids extracted from an acellular sample obtained from the human with amplification primers in a test amplification reaction; detecting reactivity of the amplification reaction, typically reactivity that is over 3 standard deviations, sometimes over 5 standard deviations, from a reference amplification reaction, wherein reactivity of over 3 is indicative of an increased risk for CJD. In some embodiments, the acellular fluid sample is serum or plasma. The nucleic acid sample can be a DNA sample or RNA sample.

Any number of primers can be used in the methods of the invention. Typically, at least one primer hybridizes to sequences in a non-coding region of the genome; often one of the primers comprises sequences that hybridize to repetitive sequences, e.g., Alu sequences, in the human genome. In some embodiments, the primers need not be from contiguous sequences or sequence on the same chromosome. In exemplary embodiments, the primers hybridize to the same sequences as the primer CHX-CJ-2F (SEQ ID NO:1) and CHX-CJ-2R (SEQ ID NO:2). Such primers can, for example, comprise at least 10 contiguous nucleotide of SEQ ID NO:1 or SEQ ID NO:2. In some embodiments, the hybridizing region of a primer comprises at least 80%, typically 90% identity to SEQ ID NO:1 or SEQ ID NO:2. Other primers that can be used in the methods of the invention are primers in the primer sets CJ_{—}1F (SEQ ID NO:3) and CJ_{—}1R (SEQ ID NO:4); CJ_{—}3F (SEQ ID NO:5) and CJ-3R (SEQ ID NO:6); and CJ_{—}5F (SEQ ID NO:7) and CJ_{—}5R (SEQ ID NO:8), or variants of such primers that hybridize to the same sequences. The primer sequences are provided in the Examples section. In some embodiments, the primers hybridize to Sequence 1 (SEQ ID NO:9), Sequence 2 (SEQ ID NO:10), or Sequence 3 (SEQ ID NO:11) of Table 2.

In typical embodiments, the amplification characteristic that is analyzed in the methods of the invention is a melting curve. The melting profile can be determined at the end of an amplification reaction or at a particular cycle number. In other embodiments, the amplification characteristic that is analyzed is a pattern on a gel, e.g., a polyacrylamide gel.

Often, the amplification reactions comprise a compound that specifically binds to double-stranded DNA, e.g, a fluorescent dye.

The invention also provides primers, and kits comprising such primers, that hybridize to sequences that are indicative of an increased risk for CJD, e.g., a primer that hybridizes to the same sequences as CHX-CJ-2F, CHX-CJ-2R, CJ_{—}1F, CJ_{—}1R, CJ3_F, CJ_{—}3R, CJ5_F, or CJ5_R. In some embodiments, a primer of the invention has at least 10 contiguous nucleotides of CHX-CJ-2F, CHX-CJ-2R, CJ_{—}1F, CJ_{—}1R, CJ3_F, CJ_{—}3R, CJ5_F, or CJ5_R; or has at least 80%, typically at least 90-95% identity to primer CHX-CJ-2F, CHX-CJ-2R, CJ_{—}1F, CJ_{—}1R, CJ3_F, CJ_{—}3R, CJ5_F, or CJ5_R. In some embodiments, the primer is CHX-CJ-2F, CHX-CJ-2R, CJ_{—}1F, CJ_{—}1R, CJ3_F, CJ_{—}3R, CJ5_F, or CJ5_R. The invention provides kits comprising such primers. A kit of the invention can also comprise various controls and reagents, including, e.g., a reference sample.

### BRIEF DESCRIPTION OF THE DRAWINGS

FIG. 1A shows a polyacrylamide gel electrophoresis (PAGE) analysis of an amplification reaction using non-coding primers (75F and 83R, Table 1), of which one is homologous to a sequence in the human prion gene. Two normal and two presumptive CJD patients were analyzed. The insert shows the band indicated by the arrow cut from the original gel and re-amplified.

FIG. 1B shows a PAGE analysis of amplification reaction products using partially degenerated primers (TC-1, A-3).

FIG. 2A shows melting curves from primers selected from sequences shown in Table 3. PCR was performed with primers CHX-CJ-2F and CHX-CJ-2R using 2 μl of CNA in a 20 μL reaction volume. FIG. 2A shows a SYBR® Green I melting curve after 30 cycles. The differences between normal individuals (N1 to N5) and CJD sera (CJD-1 through 7) were observed within a range between 82 and 90° C. CJD-1 to CJD-7: Seven individual CJD cases (CJD-1 to CJD-5, mailed-in samples; CJD-6 and CJD-7, samples drawn and processed immediately); N1 to N5: Five individual normal volunteers, samples drawn, retained at room temperature for 4 hours and processed thereafter.

FIG. 2B shows the results when comparing two runs with different preparations of primers (CHX-CJ-2F and CHX-CJ2R) as shown in FIG. 2A. The area under the curve (AUC) was calculated from melting curves after 28 cycles of PCR. Sera from all six confirmed CJD and two clinically presumptive patients were reactive in the test. All ten normals and MM controls were non-reactive.

FIG. 2C shows results from 90 sera from normal blood bank donors compared to pooled samples consisting of circulating nucleic acid (CNA) from 8 CJD patients. Samples were centrifuged no later than 4 hours at room temperature. Boxes are 5th and 95th percentile, the lines extending from the boxes represent the range of minimum to maximum AUCs.

FIG. 3 shows the results of a melting analysis at 30 cycles in a blinded study. Results from CJD samples are designated with open circles.

FIG. 4 shows an extended cycle melting curve analysis of three false positive samples at 30 cycles. The CJD positive control is designated with open circles. The false positive samples were no longer falsely positive.

FIGS. 5A and 5B shows melting curve analyses of CJD and normal samples from PCR performed using primer set 1, CJ_{—}1F and CJ_{—}1R (FIG. 5A) and primer set 3, CJ_{—}3F and CJ_{—}3R (FIG. 5B).

FIG. 6 shows the results of a melting curve analysis of a PCR performed with primer set 5, CJ_{—}5F and CJ_{—}5R.

### DETAILED DESCRIPTION OF THE INVENTION

Definitions

The term “reactivity” as used herein refers to a change in a characteristic of an amplification characteristics, e.g., a melting curve, in the presence of a nucleic acid sequence that is indicative of an increased risk for a disease, e.g., CJD. A sample has increased reactivity relative to controls when it exhibits a standard deviation of at least 1, often 2, preferably 3 or 5 relative to a reference standard.

A “positive reference” or “positive control” is a sample that is known to contain nucleic acids that are indicative of risk of a disease, e.g., CJD. In some embodiments, a “positive reference” can be from a known CJD patient that was reactive in the assay of the invention. Alternatively, a “positive reference” can be a synthetic construct that shows reactivity in an assay of the invention.

A “reference control” is a sample that results in minimal change to the amplification characteristic analyzed for the presence of nucleic acids associated with CJD. Often, such a sample is a known negative, e.g., from healthy human volunteers. For example, in diagnostic applications, such a control is typically derived from a healthy human. A “reference control” is preferably included in an assay, but may be omitted.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid.

An “amplification characteristic” refers to any parameter of an amplification reaction. Such reactions typically comprises repeated cycles. An amplification characteristic may be the number of cycles, a melting curve, temperature profile, or band characteristics on a gel or other means of post-amplification detection.

A “melting profile” or “melting curve” refers to the melting temperature characteristics of a nucleic acid fragment over a temperature gradient. In some embodiments, the melting curve is derived from the first derivative of the melting signal. The melting point of a DNA fragment depends, e.g., on its length, its G/C content, the ionic strength of the buffer and the presence of mismatches (heteroduplexes). Thus, the proportion of the molecules in the population that are melting over a temperature range generates a melting profile, which is unique to a particular fragment or population of molecules.

The term “amplification reaction” refers to any in vitro means for multiplying the copies of a target sequence of nucleic acid. Such methods include but are not limited to polymerase chain reaction (PCR), DNA ligase, (LCR), QβRNA replicase, RNA transcription-based (TAS and 3SR) amplification reactions, and nucleic acid sequence based amplification (NASBA). (See, e.g., Current Protocols in Human Genetics Dracopoli et al. eds., 2000, John Wiley & Sons, Inc.).

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; PCR Technology: Principles and Applications for DNA Amplification (Erlich, ed., 1992) and PCR Protocols: A Guide to Methods and Applications, Innis et al., eds, 1990.

The term “amplification reaction mixture” refers to an aqueous solution comprising the various reagents used to amplify a target nucleic acid. These include enzymes, aqueous buffers, salts, amplification primers, target nucleic acid, and nucleoside triphosphates. Depending upon the context, the mixture can be either a complete or incomplete amplification reaction mixture.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-25 nucleotides, in length. The length and sequences of primers for use in PCR can be designed based on principles known to those of skill in the art, see, e.g., Innis et al., supra. A primer is preferably a single-stranded oligodeoxyribonucleotide. The primer includes a “hybridizing region” exactly or substantially complementary to the target sequence, preferably about 15 to about 35 nucleotides in length. A primer oligonucleotide can either consist entirely of the hybridizing region or can contain additional features which allow for the detection, immobilization, or manipulation of the amplified product, but which do not alter the ability of the primer to serve as a starting reagent for DNA synthesis. For example, a nucleic acid sequence tail can be included at the 5′ end of the primer that hybridizes to a capture oligonucleotide. As appreciated by one of skill in the art, a primer for use in the invention need not exactly correspond to the sequence(s) that it amplifies in a hybridization reaction. For example, the incorporation of mismatches into a probe can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe, can be selected empirically using the guidance provided herein and well known in the art (see, e.g., the general PCR and molecular biology technique references cited herein).

The term “subsequence” when referring to a nucleic acid refers to a sequence of nucleotides that are contiguous within a second sequence but does not include all of the nucleotides of the second sequence.

A “non-coding” sequence refers to a sequence that is not an exon, e.g., introns, flanking regions, spacer DNA, and any of the DNA in between gene-coding DNA (intergenic DNA), including untranslated regions, 5′ and 3′ flanking regions, introns, non-functional pseudogenes, and non-functional repetitive sequences.

A “temperature profile” refers to the temperature and lengths of time of the denaturation, annealing and/or extension steps of a PCR reaction. A temperature profile for a PCR reaction typically consists of 10 to 60 repetitions of similar or identical shorter temperature profiles; each of these shorter profiles may typically define a two step or three-step PCR reaction. Selection of a “temperature profile” is based on various considerations known to those of skill in the art, see, e.g., Innis et al., supra.

A “template” refers to a double or single stranded polynucleotide sequence that comprises a polynucleotide to be amplified.

An “acellular biological fluid” is a biological fluid which substantially lacks cells. Typically, such fluids are fluids prepared by removal of cells from a biological fluid that normally contains cells (e.g., whole blood). Exemplary processed acellular biological fluids include processed blood (serum and plasma), e.g., from peripheral blood or blood from body cavities or organs; and samples prepared from urine, milk, saliva, sweat, tears, phlegm, cerebrospinal fluid, semen, feces, and the like.

“Nucleic acid” refers to a deoxyribonucleotide or ribonucleotide polymer in either single- or double-stranded form, or chimeric constructs of polynucleotides chemically linked to reporter molecules, and unless otherwise limited, would encompass known analogs of natural nucleotides that can function in a similar manner as naturally occurring nucleotides.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient, animal or human, with a disease or suspected of having a disease. Such samples include, but are not limited to, sputum, blood, serum, plasma, body cavity blood or blood products, blood cells (e.g., white cells), tissue or fine needle biopsy samples, urine, milk, peritoneal fluid, and pleural fluid, or cells therefrom. Biological samples may also include sections of tissues such as frozen sections taken for histological purposes.

An “individual” or “patient” as used herein, refers to any animals, often mammals, including, but not limited to humans, nonhuman primates such as chimpanzees and monkeys, horses, cows, deer, sheep, goats, pigs, dogs, minks, elk, cats, lagromorphs, and rodents.

A “chronic illness” is a disease, symptom, or syndrome that last for months to years. Examples of chronic illnesses in animals include, but are not limited to, cancers and wasting diseases as well as autoimmune diseases, and neurodegenerative diseases such as spongiform encephalopathies and others.

“Repetitive sequences” refer to highly repeated DNA elements present in the animal genome. These sequences are usually categorized in sequence families and are broadly classified as tandemly repeated DNA or interspersed repetitive DNA (see, e.g., Jelinek and Schmid, Ann. Rev. Biochem. 51:831-844, 1982; Hardman, Biochem J. 234:1-11, 1986; and Vogt, Hum. Genet. 84:301-306, 1990). Tandemly repeated DNA includes satellite, minisatellite, and microsatellite DNA. Interspersed repetitive DNA includes Alu sequences, short interspersed nuclear elements (SINES) and long interspersed nuclear elements (LINES).

A “rearranged sequence” or “recombined sequence” is a nucleotide sequence that is rearranged compared to normal germline DNA, i.e., the rearranged sequence is not contiguous in germline DNA in a healthy individual.

A “fragile site” is a locus within an animal genome that is a frequent site of DNA strand breakage. Fragile sites are typically identified cytogenetically as gaps or discontinuities as a result of poor staining. Fragile sites are classified as common or rare and further divided according to the agents used to induce them. For a general description of fragile sites and their classification, see, Shiraishi et al., Proc. Natl. Acad. Sci USA 98:5722-7 (2001), Sutherland GATA 8:1961-166 (1991). Exemplified sequences disclosed herein include sequences that are found in rearrangements of host genomic DNA or viral genomes that have apparently been inserted into the animal genome at a fragile site. Thus, fragile sites can contain “archived nucleic acid sequences” that are from the host and/or pathogens, including bacteria, parasites, and viruses.

The term “substantially identical” indicates that two or more nucleotide sequences share a majority of their sequence. Generally, this will be at least about 80%, 85%, or 90% of their sequence and preferably about 95% of their sequence. The percent identity can be determined using well know sequence algorithms or by manual inspection. Another indication that sequences are substantially identical is if they hybridize to the same nucleotide sequence under stringent conditions (see, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997). Stringent conditions are sequence-dependent and will be different in different circumstances. Generally, stringent conditions are selected to be about 5° C. (or less) lower than the thermal melting point (Tm) for the specific sequence at a defined ionic strength and pH. The Tₘ of a DNA duplex is defined as the temperature at which 50% of the nucleotides are paired and corresponds to the midpoint of the spectroscopic hyperchromic absorbance shift during DNA melting. The Tₘ indicates the transition from double helical to random coil.

Typically, stringent conditions will be those in which the salt concentration is about 0.2×SSC at pH 7 and the temperature is at least about 60° C. For example, a nucleic acid of the invention or fragment thereof can be identified in standard filter hybridizations using the nucleic acids disclosed here under stringent conditions, which for purposes of this disclosure, include at least one wash (usually 2) in 0.2×SSC at a temperature of at least about 60° C., usually about 65° C., sometimes 70° C. for 20 minutes, or equivalent conditions. For PCR, an annealing temperature of about 5° C. below Tm, is typical for low stringency amplification, although annealing temperatures may vary between about 32° C. and 72° C., e.g., 40° C., 42° C., 45° C., 52° C., 55° C., 57° C., or 62° C., depending on primer length and nucleotide composition. High stringency PCR amplification, a temperature at, or slightly (up to 5° C.) above, primer Tm is typical, although high stringency annealing temperatures can range from about 50° C. to about 72° C., and are often 72° C., depending on the primer and buffer conditions (Ahsen et al., Clin Chem. 47:1956-61, 2001). Typical cycle conditions for both high and low stringency amplifications include a denaturation phase of 90° C.-95° C. for 30 sec-2 min., an annealing phase lasting 30 sec.-10 min., and an extension phase of about 72° C. for 1-15 min.

Nucleic Acids Detected in the Methods of the Invention

The invention provides a method for detecting circulating nucleic acid (CNA) associated with CJD using primers in an amplification reaction. Nucleic acid molecules detected in the methods of the invention may be free, single or double stranded, molecules or complexed with protein or lipid. RNA molecules need not be transcribed from a coding gene, but can be transcribed from any sequence in the chromosomal DNA. Exemplary RNAs include small nuclear RNA (snRNA), mRNA, tRNA, rRNA, microRNA (miRNA), and interference RNA (iRNA).

The nucleic acid molecules may comprise sequences transcribed from repetitive sequences in the genome of the individual from which the sample is derived. The detected nucleic acid molecules may also be the products of rearrangement of germline sequences and/or exogenous sequences introduced into the genome, e.g., exogenous viral sequences.

The method does not require knowledge of the polynucleotide sequences present in the test samples to be evaluated. Thus, a polynucleotide detected using this method may be a particular polynucleotide or may be a population of polynucleotides that are present in the sample. Furthermore, even in instances, where the polynucleotide to be detected has a known sequence, the polynucleotide in a particular sample need not have that sequence, i.e., the sequence of the polynucleotide in the sample may be altered in comparison to the known sequence. Such alterations can include mutations, e.g., insertions, deletions, substitutions, and various rearrangements.

Test Samples

The test samples are typically from any source, but are typically biological samples. In some embodiments, the biological samples are blood samples, such as those obtained from a blood bank. Such samples can be samples from a particular individual, or from a pooled sample from multiple individuals. Thus, detection of an individual at increased risk for CJD encompasses embodiments in which the sample from the individual is present in a pooled sample. The identity of the individual need not be known. Biological samples are not limited to blood samples but can be from any source.

In some embodiments, the biological sample is obtained from a patient who is to undergoing surgery. Although frequently the test is performed on serum or plasma, it may also be performed on other acellular fluids. In some embodiments, a sample, for example serum or plasma, can be additionally processed, e.g., by centrifugation, filtration, and by other physical or chemical means.

Target nucleic acid can be from any source, but is typically a sample that comprises small quantities of nucleic acid, e.g., nucleic acid samples obtained from acellular fluids that are not readily quantified by standard PCR methodology. In particular embodiments, the test sample is a nucleic acid, e.g., RNA or DNA that is isolated from serum or plasma.

Amplification Reactions

Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Amplification reactions to amplify the nucleic acids in the samples are performed using standard methodology. The test sample to be evaluated is included early, typically at the onset, of the amplification reaction. Typically, the amplification reaction is a PCR.

The primer pairs to be used in the PCR often include at least one primer that is from a non-coding region. In some embodiments, a primer may hybridize to a sequence that comprises repetitive elements, e.g., Alu or SINE sequences, or sequences involved in rearrangements. In some embodiments, the individual primers in a primer pair need not hybridize to sequences that are present on a same un-rearranged chromosome. For example, a primer pair may amplify a sequence that results from chromosomal rearrangement. The ability of such primers to amplify nucleic acids that are indicative of risk for CJD can be determined empirically.

In one embodiment, the primers are CHX-CJ-2F, 5′-GGATTCCACTGCACTCCA-3′ (SEQ ID NO:1) and CHX-CJ-2R, 5′-CAGTTGCTGTGTAGCTATCCCTTT-3′ (SEQ ID NO:2). As understood by one of skill in the art, CHX-CJ-2F and CHX-CJ-2R sequences can be modified such that they still amplify the sequences of interest. For example, at least 3 nucleotides can be changed in the sequences. In some embodiments, a primer for use in a method of the invention to detect CJD CNA comprise at least 10 contiguous nucleotides; often at last 11, 12, 13, 14, 15, 16, 17, or 18 contiguous nucleotides; or 19 or more contiguous nucleotides of SEQ ID NO:1 or SEQ ID NO:2. In other embodiments, such a primer is at least 80% identical, often 90% or 95% identical to SEQ ID NO:1 or SEQ ID NO:2.

In other embodiments, other primers that amplify the sequence shown as Sequence 2 in Table 2 can be used in the methods of the invention. Such primers are designed using criteria well known in the art. Similarly, primers that amplify the sequences shown as Sequence 1 and Sequence 3 in Table 2 may be used for the detection methods of the invention. For example, primers such as CJ_{—}1F and CJ_{—}1R, CJ_{—}3F and CJ_{—}3R, and CJ_{—}5F and CJ_{—}5R can be used to assess risk for CJD. Conservative variants of these primers, which retain the ability to amplify the sequence of interest, an also be used. Such variants include primers that comprise at least 10 contiguous nucleotides; often at least 11, 12, 13, 14, 15, 16, 17, or 18 contiguous nucleotides; or 19 or more contiguous nucleotides of primer CJ_{—}1F, CJ_{—}1R, CJ_{—}3F, CJ_{—}3R, CJ_{—}5F, or CJ_{—}5R

Amplification reactions can be evaluated using any known techniques. The amplification reaction can be monitored either during amplification or at the end of amplification. The reaction is monitored using various known techniques. In one embodiment, the amplification reaction mixture comprises doubled-stranded specific nucleic acid dyes. These dyes, typically fluorescent, specifically intercalate into double-stranded nucleic acids relative to single-stranded nucleic acid molecules. Accordingly, they can be used to monitor the amount of double-stranded nucleic acid present during various stages of the amplification reaction or at the end of the amplification reaction. Such dyes include SYBR® Green I and Pico Green.

Various characteristics of the test nucleic acid amplification reaction can be changed by the presence of the nucleic acids associated with CJD CNA. These include cycle number changes and melting curve parameters. These endpoints can be measured during the amplification reaction or at the end of the amplification reaction. For example, in using cycle number as an endpoint parameter, the amount of product generated/cycle can be assessed during the reaction. The presence of nucleic acids associated with an increased risk for CJD is detected by an alteration in the amount of product generated through the course of the reaction, or alternatively, at a particular cycle number.

Other methods for measuring differences in the products of an amplification reaction include known techniques such as oligonucleotide probing on solid phase (e.g., arrays) or in liquid phase (e.g., Taqman) in various techniques, single strand conformation polymorphism (SSCP) mass-spectrometry, e.g., MALDI-TOF, ESI-QTOF, APCI-TQMS or APCI-MSn, electrophoresis, e.g., capillary electrophoresis such as that performed for microsatellite analysis, or denaturing HPLC, combined with either UV, fluorescence, or using an ESI or APCI interface with any mass spectrometry detection, which can also be done with capillary electrophoresis separation.

In some embodiments, melting curve analysis is used as an endpoint for analysis of the test nucleic acid samples.

Determination of Test Sample Melting Curve Parameters

The conditions for defining whether a sample is considered to be reactive, i.e., the sample contains CJD nucleic acid sequences, vs. whether it is considered to be unreactive is established by running negative and positive controls samples. The maximum signal separation between negative and positive references are determined, i.e., temperatures are identified that can be used for area under the curve ratios that give maximum separation between the negative and positive controls. Typically, the area under the curve analysis is performed at a range of 82° C. to 90° C. This range is used as it is not prone to the influence of non-specific products, e.g., primer-dimers, which frequently may be present. Similarly, cycle number is selected based on the maximum separation achieved in comparing known negative and positive samples. Thus, the optimal conditions for the amplification and area under the curve analysis can be determined for a particular primer set used to determine reactivity. Reactivity of each individual sample is calculated on the basis of an area under the curve above the detection limit, which is defined as mean +3, typically mean +5, standard deviations above baseline of non-template or reference controls. One of skill will appreciate that other amplification parameters, such as annealing temperature, can also be optimized similarly, e.g., by selecting annealing temperature based on the maximum separation achieved in comparing known positive and negative samples.

Various controls are often included in the reaction. These include a non-template control, samples from known healthy animals, and a known positive control. A non-template control is a reaction in which non sample is added to the PCR. The reference controls are samples known to be normal, e.g., from humans known to be negative for CJD CNA. The positive control may be an artificial positive control or can be from a source that is known to have CJD CNA, e.g., a CJD patient. An assay need not contain all of the controls. Further, some control values, e.g., a reference control, can be supplied from previously performed assays.

As explained above, the presence of the nucleic acid associated with CJD risk is determined by a significantly different alteration in the test sample, as compared to the references standards.

Analysis of Melting Curve for Test Samples

The test samples are normally run concurrently with the positive and references controls. This monitoring will provide a melting profile of the amplified product. Typically, the Tm is obtained in a separate melting process at the end of the amplification cycle, during which fluorescence is continuously monitored and a melting profile is obtained. However, a Tm may also be obtained at some point during the amplification process.

Fluorescence monitoring is generally used to produce the melting curves. For example, double-stranded-specific DNA specific dyes, e.g., SYBR® Green, can be incorporated into the amplification reaction or added to the reaction only for detection purposes after the amplification. Thus, specific probe is not required to monitor the reaction. SYBR® Green dye is thought to bind within the minor groove of dsDNA; thus the fluorescent signal steadily decreases as the dsDNA melts into single strands. Typical melting curve analyses are described in the following examples.

As noted above, analogous methodology can be employed to determine the endpoint standards for any amplification parameter to be tested. For example, the cycle number in the presence of positive and negative reference standards is determined and the mean and standard deviations calculated to select a cutoff value for whether a sample is considered to be reactive or unreactive. Further, analyses such as electrophoresis can be used to assess the presence of reaction products.

In some embodiments, the detection methods of the invention detect the presence of the sequences set forth in Table 2 (sequence 1 (SEQ ID NO:9), sequence 2 (SEQ ID NO:10), or sequence 3 (SEQ ID NO:11)) in an acellular sample from a patient at risk for CJD. Such detection methods include, for example, amplification methods as described herein and hybridization based assays such as microarray analysis and the like using polynucleotide sequences as probes that hybridize to SEQ ID NO:9, 10, or 11. Such detection methodology is well known in the art (see, e.g., Sambrook and Ausubel, both supra).

### EXAMPLES

### Example 1

### Detection of Individuals at an Increased Risk for CJD

This example describes detection of CJD CNA. In summary, a PCR using non-coding region primers in a differential display approach was used on the sera from six confirmed cases of CJD, two presumptive clinical cases of CJD and eight healthy, laboratory volunteers. All eight sera from CJD confirmed and two presumptive cases were reactive in the assay. None of the eight healthy volunteer samples were reactive. Cloning of the reactive PCR products revealed human germ-line rearranged sequences. All sequenced PCR CNA fragments from CJD patients shared a 27-mer base sequence homologous to the Alu consensus sequences.

Experimental Protocol

Human subjects: Sera from CJD patients were obtained from the laboratory of Dr. Walter Schulz-Schaeffer. Sera from normals were drawn from ten volunteers.

Serum collection: Special care was taken in collection, processing and storage of serum samples. Blood from a suitable vein was drawn into tubes containing a coagulation accelerator manufactured for serum preparation. Until further processing, the tubes were stored at room temperature for not longer than 4 hours. Centrifugation was done at 2-8° C., 1000× g for 15 min. The serum supernatant was transferred into 1.5 mL microcentrifuge cups in 0.5 mL aliquots and frozen immediately at −20° C. or −80° C. until use.

Preparation of serum fractions: Frozen serum was thawed at 4° C. in an ice-water bath and 250 μL were transferred into a 1.5 mL microcentrifuge tube. The tube was centrifuged at 4,000× g for 35 min at 4° C. in a Model 5214 bench top centrifuge (Eppendorf, Hamburg, Germany). The supernatant was carefully removed and transferred into a new tube, which was subjected to a subsequent centrifugation at 20,000× g for 30 min at 4° C. the pellet was used for further analyses.

Nucleic acid extraction: 20,000× g pellets were used with a standard silica-based nucleic acid extraction (NucleoMag Kit; Cat#: 744500.24, Macherey-Nagel, Düren, Germany) according to the manufacturer's instructions. Briefly, the pellet was resuspended in 125 μL Lysis-buffer and incubated for 10 min. Consecutively, magnetic beads in binding buffer were added, supernatant was removed and the magnetic beads were washed three times using the appropriate washing solutions. Nucleic acids were eluted with 30 μL elution buffer at 58° C. The resulting nucleic acid (NA) solutions were either used immediately or frozen at −80° C. until further use.

CJD-enriched sequences: CJD-enriched gene sequences were cloned and sequenced from sera collected from patients with presumptive CJD that were confirmed post mortem as CJD-positive. Two healthy human sera were used for comparison. A set of oligonucleotide primers derived from the non-coding region of the PrP (prion) gene and from conserved repetitive elements and a second set of primers with partially degenerate sequences was used for differential display. The latter primers contained a modified T7 signal sequence (5′) followed by a unique stem sequence of 4 to 6 bp, followed by a stretch of 4N and one final unambiguous base. Primers were used in multiple combinations. 2 μL extracted NA was used as template in 20 μL PCR (Advantage-2 PCR Kit, BD-Clontech, Heidelberg, Germany), with 30 to 35 cycles at 48 to 55° C. annealing (60 sec), 68° C. extension (2 min), 94° C. denaturation (1 min). Samples from CJD confirmed cases and healthy control individuals were loaded side-by-side on a PAGE gel and analyzed as described. Clearly differentially expressed bands were cut out of gels, eluted and subjected to re-amplification with either the specific primers or T7 primers where appropriate using Taq polymerse. The products were purified and ligated into a linearized TA-vector. Ligation was performed overnight at 4° C. using 1U T7 DNA ligase, 1 μg of the vector and the PCR product prepared as described above. The product was transformed into electro competent E. coli (Dh10b) and plated on AXI LB agar (ampicillin, X-Gal, IPTG). After overnight incubation at 37° C., positive (white) clones were picked and cultured in 5 mL LB-medium with ampicillin. Bacteria were harvested and plasmids were isolated according to standard protocols, and reconstituted in 50 μL TBE buffer. The plasmids were sequenced using M13 forward and M13 reverse primers with a model 3100 ABI capillary sequencer using unlabelled primers with big-dye-termination.

Sequence comparison: Genetic analysis was applied to the sequences using the Sequencer™ program. All sequences from cloning were imported and subjected to a vector trim algorithm. The resulting inserts were assembled and the resulting overlapping contigs checked for their origin. Contigs in which only clones derived from CJD patients were present, were selected for primer design.

Diagnostic PCR. Two μL of the extracted NA from serum fractions were used in a PCR in a total volume of 20 μL. Primers CHX-CJ-2F and CHX-CJ-2R, were used at 1 μM each using a proof reading polymerase system (Advantage-2 PCR Kit, BD-Clontech, Heidelberg, Germany). After either 25 or 28 cycles of 95° C. for 30 sec, 68° C. for 105 sec, a SybrGreenI (Cat#: S7563, Molecular Probes, Eugene, Oreg., USA) derived melting curve was recorded in a MX4000 PCR system (Cat#: 401260, Stratagene, La Jolla, Calif., USA). The area under the curve of the derived melting function −d(F)/dT between 85° C. and 90° C. was used for analysis. This range was used as it was not prone to the influence of non-specific products, e.g., primer-dimers, which frequently may be present due to the use of SybrGreenI during PCR. Reactivity of each individual sample was calculated on the basis of an area under the curve (AUC) above the detections limit, which can be defined, e.g., as mean +5 standard deviations above baseline of non-template or reference controls.

Statistical analysis. The proportion of reactivity in the CJD groups and the healthy control groups was calculated. The statistical significance between the CJD groups and healthy controls was estimated using the Chi-square test.

PAGE: Three μL of the PCR mixture was mixed with loading buffer and applied to a precast 12-20% polyacrylamide gel in TBE buffer (45 mM Tris, 45 mM boric acid, 1 mM EDTA) (Novex 4-20% TBE Gel; Cat#: EC62255, Karlsruhe, Germany). Electrophoresis was run at ambient temperature for 45 to 55 min at 210 V. The gels were stained for 20 min in a SybrGold (Cat#: S11494, Molecular Probes, USA) solution and were photographed under UV light.

Results

Sera from two presumptive CJD patients and two apparently healthy human individuals were used for initial analysis. After preparation of serum nucleic acid as described above, PCRs were performed for 35 cycles at denaturation at 95° C. for 30 seconds, annealing at 55° C. for 30 seconds, elongation at 68° C. for 1 min. The primers used (in various combinations) are shown in Table 1.

TABLE 1

Sequences Selected from Non-coding regions of
Human Prion Gene

| | |
|---|---|
| 75F | CCACTGCACTCCAGCCTG |
| 75R | CAGGCTGGAGTGCAGTGG |
| 76R | GGTCTCCAGGTCTGTTGGATC |
| 77F | CACACTGATATGCCTTATGCGC |
| 77R | CCGCATAAGGCATATCAGTGTG |
| 78R | CCTCCACTTTATTGAGCACTTAG |
| 79F | CTCACATAAACATGGCCCAGGC |
| 80F | GCATCTAAGTGGGCTTAGCACTG |
| 81R | GATTGAACTCAATTATGTTTATGC |
| 82R | GCTGGTCTCAAACTCCTCACCTC |
| 83R | CTACACAGTTGCTGTGTAGC |

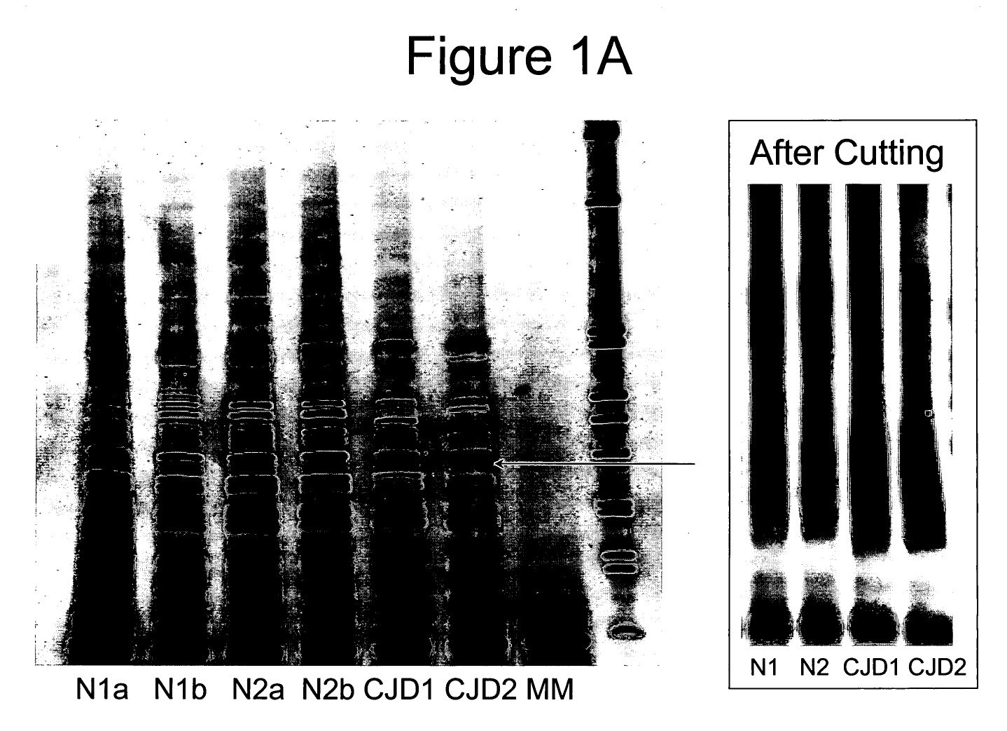

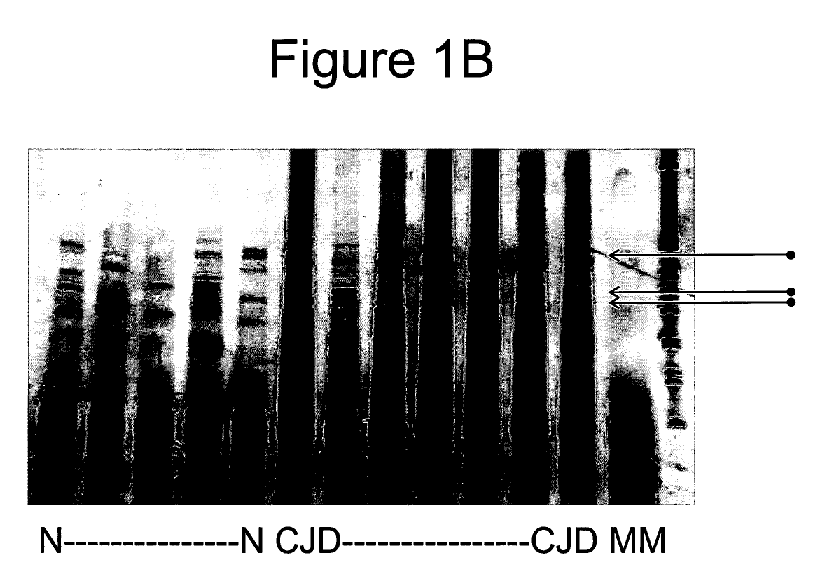

The resulting PCR products were separated on a PAGE and investigated for differentially expressed bands. One primer combination (75F/83R) revealed a band at approx. 280 bp that was only present in CJD patients, but not in normal controls, as given in FIG. 1A. The respective band was excised and re-amplified with the same set of primers. The resulting PCR products were subjected to PAGE analysis and used for direct TA cloning by means of the Promega p-Gem-T vector system.

The resulting PCR products from two CJD cases were cloned and sequenced as described. Three sequences were found to be present in both presumptive CJD cases, but not present in either normal individual. These data indicate that nucleic acid sequences may be involved in discriminating between normal and CJD patients.

TABLE 2
Sequences derived from PCR products using Primers 75F/83R

Sequence 1 (SEQ ID NO:9)
ACTATAGAATACTCAAGCTATGCATCCAACGCGTTGGGAGCTCTCCCATATGGTC
CACTTGCAGGCGGCCGCACTAGTGATTCCACTGCACTCCAGCCTGGGTGACAGAA
TGACACTGTTTCTAAAAAAACAAAACAAAACAAAACAAAAAAAATTCTGCATTT
TTTTATAAGGATCTGCTTTAACTCTAACTGCTCCTGGAAATAAGCCCTGACTAATC
AAGGCTACACAGCAACTGTGTAGAATCCCGCGGCCATGGCGGCCGGGAGCATGC
GACG
Sequence 2 (SEQ ID NO:10)
GTAAAACGACGGCCAGTGAATTGTAATACGACTCACTATAGGGCGAATTKRGCC
MGRCGATSGSMKGCTCCYGKCCGGCSMTGGCCGCGGGATTCCACTGCACTCCAG
CCTGGGCGACAGAGCAAGACTCCATCTCAAAAAACAAACAAAAAACAATCATAT
GATCCAGCAATCCCACTACTGGGAATTYATGGAAAGGAAAAGAAATCAGTGTAT
CAAAGGGATAGCTACACAGCAACTGTGTAGAATCACTAGTGCGGCCGCCTGCAG
GTCGACCATATGGGAGAGCTCCCAACGCGTTGGATGCATAGCTTGAGTAT
Sequence 3 (SEQ ID NO:11)
GGCGAGCCTGTCCGGCGCTGGCCGGGATCACCCTTBTGGAGCATTGTGAACTCTC
AAGCTTTATTTTCTAATCTGAAAATGAGGGAGAACAGTAACTACCTTATAGGTTA
TGTGGATTAAATGAGATAGTGCCCCGTCAATCTTCACTATATATTAGTTATAATC
ATTTTTTTTTTCTTTGAGACAGGGTCTCATTCTGTCACCCAGGCTGGAGTGCAGTG
GAATCACTAGTGCGGCCGCCTGCAGGTCGACCATATGGGAGAGCTCCCAACGCG
TTGGATGCATAGCTTGAGTATTCTATAGTGTCACCTA

DNA sequence alignments (5′ to 3′, left to right) from the three individual CNA fragments derived from PCR with 75F and 83R primers are shown below. A common homology in all three CNA fragments is homologous to Alu sequences, as shown below.

| Sequence 1 (Alu-Sx): | | | |
|---|---|---|---|
| >gnl\|alu\|X68195_HSAL000932 (Alu-Sx) | | | |
| Length = 186 | | | |
| Score = 46.1 bits (23), Expect = 2e−07 | | | |
| Identities = 26/27 (96%) | | | |
| Strand = Plus/Plus | | | |
| Query: | 83 | ccactgcactccagcctgggtgacaga | 109 |
| | | \|\|\|\|\|\|\|\|\|\|\|\|\|\|\|\|\|\|\|\|\|\|\|\|\|\|\| | |
| Sbjct: | 124 | ccactgcactccagcctgggggacaga | 150 |

| Sequence 2 (ALU-Sx) | | | |
|---|---|---|---|
| >gnl\|alu\|X68195_HSAL000932 (Alu-Sx) | | | |
| Length = 186 | | | |
| Score = 76.9 bits (37), Expect = 1e−16 | | | |
| Identities = 40/41 (97%) | | | |
| Strand = Plus/Plus | | | |
| Query: | 93 | ccactgcactccagcctgggcgacagagcaagactccatct | 133 |
| | | \|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 124 | ccactgcactccagcctgggggacagagcaagactccatct | 164 |

| Sequence 3 (ALU-J) | | | |
|---|---|---|---|
| >gnl\|alu\|X68195_HSAL000932 (Alu-Sx) | | | |
| Length = 186 | | | |
| Score = 46.2 bits (23), Expect = 2e−07 | | | |
| Identities = 26/27 (96%) | | | |
| Strand = Plus/Minus | | | |
| Query: | 196 | tctgtcacccaggctggagtgcagtgg | 222 |
| | | \|\|\|\|\|\| \|\|\|\|\|\|\|\|\|\|\|\|\|\|\|\|\|\|\|\| | |
| Sbjct: | 150 | tctgtcccccaggctggagtgcagtgg | 124 |

For the sequences above, primers were designed to sequences 1, 2, and 3 to selectively amplify the given CNA. Primers are listed in Table 3. The numerical designation refers to the sequence number.

TABLE 3

Primers selected from sequences shown in TABLE 2.
Primers are 5′ to 3′.

| | | |
|---|---|---|
| CJ_1F | AGCCTGGGTGACAGAATGAC | (SEQ ID NO:3) |
| CJ_1R | TCAGGGCTTATTTCCAGGAG | (SEQ ID NO:4) |
| CJ_2F | GGATTCCACTGCACTCCA | (SEQ ID NO:1) |
| CJ_2R | CAGTTGCTGTGTAGCTATCCCTTT | (SEQ ID NO:2) |
| CJ_3F | GCATTGTGAACTCTCAAGCTTTATT | (SEQ ID NO:5) |
| CJ_3R | CCTGGGTGACAGAATGAGAC | (SEQ ID NO:6) |
| CJ_5F: | TCTCAAGCTTTATTTTCTAATCTGA | (SEQ ID NO:7) |
| CJ_5R: | AACTAATATATAGTGAAGATTGACG | (SEQ ID NO:8) |

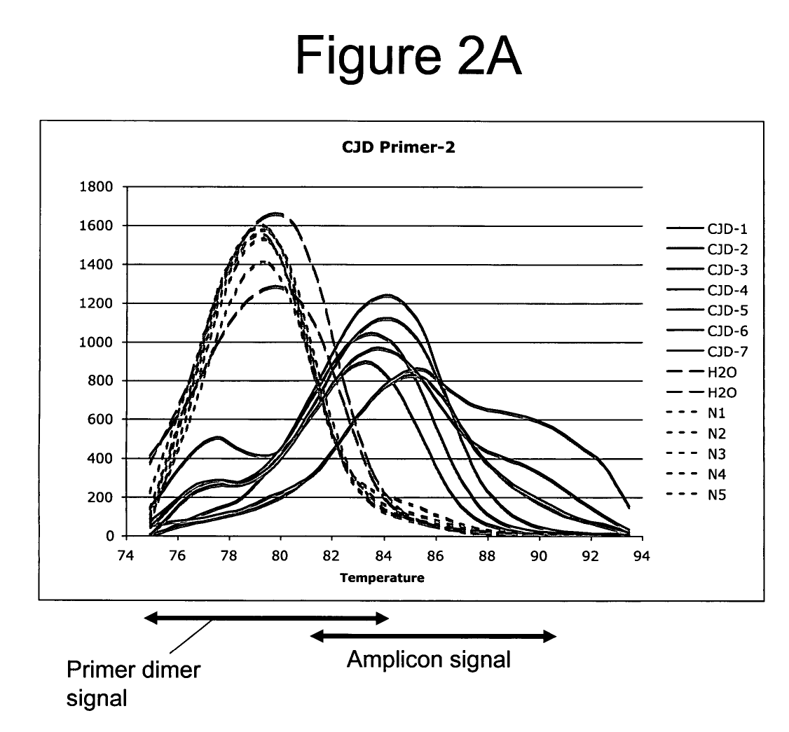

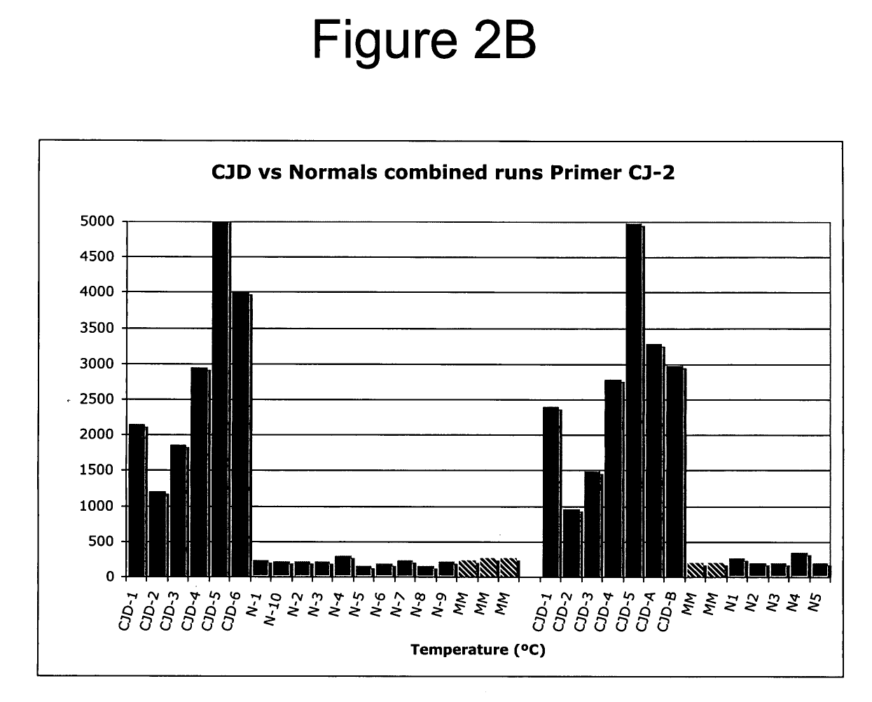

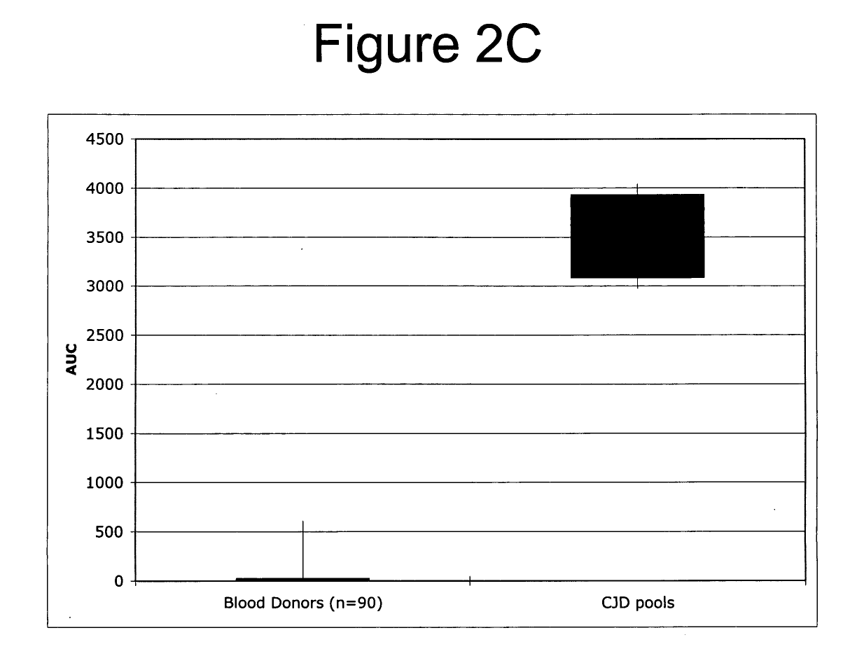

Melting curve analysis using the CHX-CJ-2F/CHX-CJ-2R primers was performed on confirmed CJD samples and normal controls (FIG. 2A through FIG. 2C). The primers were derived from Sequence 2 (Table 2). FIG. 2A shows melting curves from PCR (30 cycles). The differences between normal individuals (N1 to N5) and CJD sera (CJD-1 through 7) were observed within a range between 82° C. and 90° C. FIG. 2B shows the results when comparing two runs with different preparations of primers (CHX-CJ-2F and CHX-CJ2R) as shown in FIG. 2A. The area under the curve (AUC) was calculated from melting curves after 30 cycles of PCR. Sera from six confirmed CJD and two clinically presumptive patients were reactive in the test. All ten normal samples and MM controls were non-reactive. Additional samples from healthy normal controls were evaluated. The results of melting curve analysis of 90 normal samples in comparison to the samples from known CJD cases are shown in FIG. 2C. The y-axis shows the AUC. None of the 90 normal samples showed reactivity in the melt curve analysis. A sample is considered to be reactive when it exhibits a Z-value of over 3 standard deviations from reference.

### Example 2

### Blind Study Detecting CJD Samples

In a blind study to detect CJD, 10 blinded samples were evaluated. Samples were kept frozen until the day of nucleic acid extraction. The following samples were used for CNA extraction and polymerase chain reaction (PCR):

10 unknown plasma samples (labeled CJ-2-99-126, CJ-2-99-152, CJ-2-99-153, CJ-2-99-176, CJ-2-99-177, CJ-2-99-178, CJ-2-99-181, CJ-2-99-192, CJ-2-99-206, CJ-2-99-214;

2 archived Chronix Biomedical positive CJD samples (Pos Control-125, Pos Control-144); and

EDTA-plasma samples from 2 healthy blood donors (CJ-2-New-Ch, CJ-2-New-Ju).

Frozen plasma or serum was thawed at 4° C. in an ice-water bath and 200 μL were transferred into a 1.5 mL microcentrifuge tube. The tube was centrifuged at 4,000× g for 25 min at 4° C. in a Model 5214 bench top centrifuge (Eppendorf, Hamburg, Germany) to remove cell debris. The supernatant was transferred into a fresh tube and subjected to 35 min centrifugation at 20,000× g. The supernatant was carefully removed and the pellet was used for further analyses.

For the nucleic acid extraction, 20,000× g pellets were used with a standard silica based nucleic acid extraction (NucleoMag Kit, Macherey und Nagel, Düren, Germany) according to the manufacturer's instructions. The resulting nucleic acid solutions were immediately frozen at −80° C. until further use.

PCR

Extracted nucleic samples were subjected to PCR using primers CHX-CJ-2F and CHX-CJ2R. The resulting PCR products melted over a temperature range of 81.5° C. and 94° C. Two runs were performed although there was not enough sample to rerun CJ-2-99-126. The PCR analysis was performed as follows. Two μL of the extracted NA from plasma and serum fractions were used in a total PCR reaction volume of 20 μL. Primers CHX-CJ-2F and CHX-CJ-2R (Cat#: 42-51/0704 and 42-52/1003, Chronix Biomedical GmbH, Göttingen, Germany) were used at 0.5 μM each using a proofreading polymerase system (Advantage-2 PCR Kit, BD-Clontech, Heidelberg, Germany). After 28 cycles of 95° C. for 30 sec, 58° C. for 45 sec, 68° C. for 1 min, a SybrGreenI (Cat#: S7563, Molecular Probes, Eugene, Oreg., USA) derived melting curve was recorded in a MX4000 PCR system (Cat#: 401260, Stratagene, La Jolla, Calif., USA). The first run was prepared as described above, where all samples were run in duplicate. For the second run, sample and total reaction volume was reduced by half to conserve samples. A second independently prepared CNA extraction (exception: CJ-2-99-126 was “quantity not sufficient”) was run again in duplicate. The PCR products resulting from the amplification reactions melted over a temperature range of 81.5° C. to 94° C.

Methods of Evaluation

The area under the curve (AUC) was derived from melting function −d(F)/dT using either of two temperature ranges. Method 1 utilized the temperature range between 82.5° C. and 88.5° C. Method 2 utilized the temperature range between 89° C. and 92.5° C. Reactivity of each individual sample was calculated on the basis of an AUC and standardized based on the negative controls. Z-values are used as the method of determining statistical significance and were calculated for each sample and method using the formula:

$$\begin{matrix}{Z}_{i} = \frac{{AUC}_{i} - \overline{{AUC}_{N}}}{SD ( {AUC}_{N} )} \\ {Z}_{i} = Z \text{-} value of a sample \\ {AUC}_{i} = AUC of a sample \\ SD ( {AUC}_{N} ) = Standard deviation of AUC in Normals \\ \overline{{AUC}_{N}} = Geometrical mean of AUC in Normals\end{matrix}$$

The Z values from the AUC for 2 runs using two different methods of calculations were calculated. The Z-values are shown in Table 4.

TABLE 4
Z-Values Using Two Different Melting Curve Ranges

Z-Values Using Two Methods of Calculation

| Samples | Method 1 | Method 2 |
|---|---|---|
| Positive Control | 11 | 191 |
| Negative Control | 0 | 0 |
| CJ-2-99-214 | 13 | 18 |
| CJ-2-99-153 | 13 | 136 |
| CJ-2-99-126 | 10* | 11* |
| CJ-2-99-206 | 8 | 300 |
| CJ-2-99-152 | 7 | 334 |
| CJ-2-99-178 | 3 | 145 |
| CJ-2-99-181 | 2 | 1 |
| CJ-2-99-177 | 1 | 2 |
| CJ-2-99-176 | 1 | 1 |
| CJ-2-99-192 | −1 | 0 |

*Only two data points used

The samples evaluated were the blinded samples, two samples from confirmed CJD cases, and two samples from healthy donors. Samples demonstrated one of three patterns: low reactivity, CJD reactivity with a peak at 83° C., and CJD reactivity with a peak at 92° C. Only one sample (CJ-2-99-153) showed either reactive pattern in each of the runs. The CNA testing format involves calculating the Area Under the Curve (AUC) for a temperature range determined by trials. In the absence of a large clinical study, both calculations are presented. Table 4 summarizes the combined results of two runs. The Z-values were consistently below 2 for four of the samples regardless of the temperature used to calculate the AUC: CJ-2-99-176, CJ-2-99-177, CJ-2-99-181 and CJ-2-99-192. The Z-values were consistently higher than 5 for five of the samples regardless of the temperature used to calculate the AUC: CJ-2-99-126, CJ-2-99-152, CJ-2-99-153, CJ-2-99-206 and CJ-2-99-214. One sample, CJ-2-99-178, was reactive in one run and had very low reactivity in the second run.

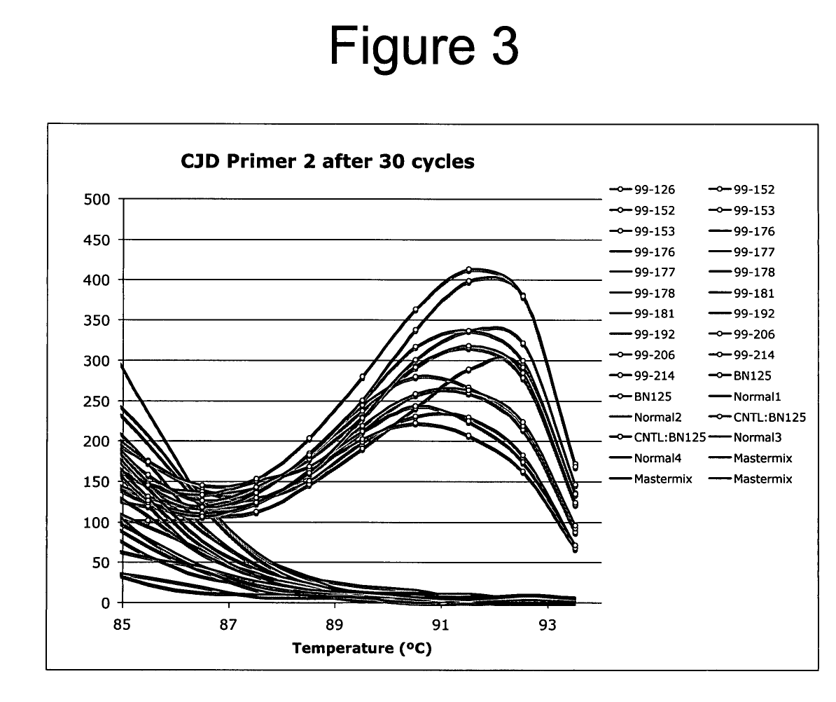

The results above were obtained from the melt at 28 cycles. Two temperature range calculations were provided since more data are required to select the final result interpretations. All the original data from the previous runs including the melting curve from 30 cycles had been retained. FIG. 3 shows data generated from Run II at 30 cycles. The data show that sample CJ-2-99-178 is not reactive. The Method 2 range (89° C. and 92.5° C.) is the preferred range in this example to select for the best separation of reactive and non-reactive samples.

The blinded study identified all of the CJD samples (numbers 214, 153, 126, 206, and 152).

### Example 3

### Evaluation of False-positive Samples

CNA Test for CJD

In Example 2, results of the melting curve after 28 cycles and 30 cycles of amplification were presented. This example presents PCR data comparing 28 and 30 cycles from a new run of retained frozen, extracted DNA from false-positive samples.

Additional Samples

Blood donors: Ninety-two samples from blood donors were provided by the Blood Bank of the University Clinics Göttingen (UKG). These samples were divided into groups and stored for various recorded times at RT before serum separation. One additional group was kept cold before centrifugation.

Frozen retained samples: The following samples were used for CNA extraction and polymerase chain reaction (PCR). Samples 425157, 421559 and 421563 were the three serum samples from Göttingen's Universitätsklinik Blood Bank that had shown false-positive reactions in the CJD blood test. The retained extracted DNA (extraction performed as in Example 2) from these false-positive samples was kept frozen at −80° C. and used for further amplification. The positive control for this analysis was frozen retained extracted DNA from CJD confirmed case BN125. Frozen retained extracted DNA was thawed on ice and used directly in the PCR.

PCR

One μL of the thawed retained, extracted DNA was used in a total PCR reaction volume of 10 μL. Primers CHX-CJ-2F and CHX-CJ-2R (Cat#: 42-51/0704 and 42-52/1003, Chronix Biomedical GmbH, Göttingen, Germany) were used at 0.5 μM each using a proofreading polymerase system (Advantage-2 PCR Kit, BD-Clontech, Heidelberg, Germany). After 28 and 30 cycles of 95° C. for 30 sec, 58° C. for 45 sec, 68° C. for 1 min, a SybrGreenI (Cat#: S7563, Molecular Probes, Eugene, Oreg., USA) derived melting curve was recorded in a MX4000 PCR system.

Results

Pre-Analytics

Ninety-two samples were obtained from the UKG Blood Bank. Samples were stored under different conditions before serum separation. Time at RT before centrifugation was recorded for each sample. Table 5 shows that false positive reactions began to occur at >4.5 hrs. Samples stored at 4° C. to 8° C. prior to centrifugation did not show any increased reactivity.

TABLE 5

| Hours at Room Temperature | N | Number Repeatedly Reactive |
|---|---|---|
| 0-1 | 24 | 0 |
| 1-2.5 | 24 | 0 |
| 2.5-4.5 | 31 | 0 |
| >4.5 | 13 | 3 |

Increasing Cycle Numbers

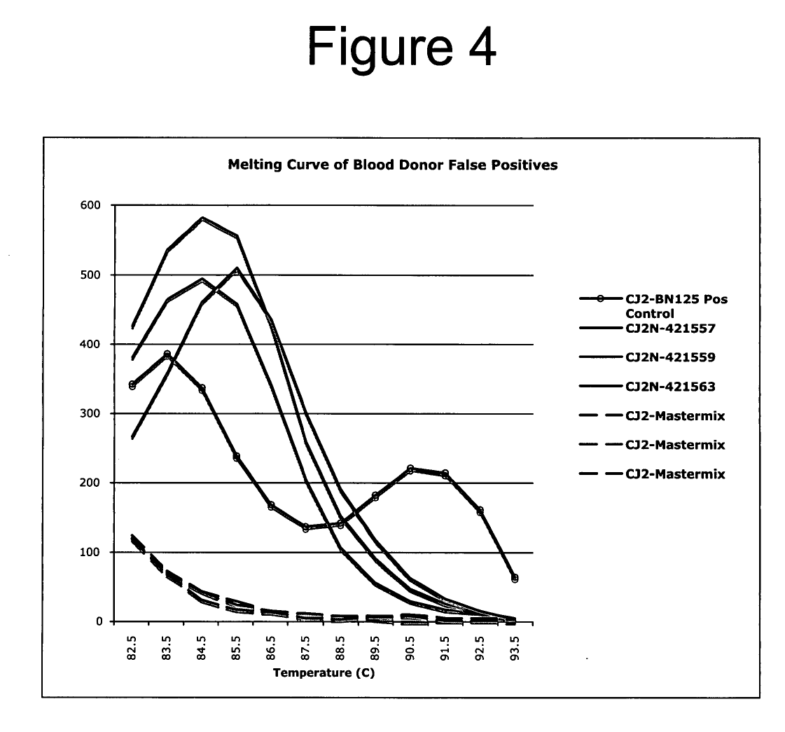

In order to minimize the contributions of pre-analytic processing, the frozen, retained extracted DNA from all three false-positive samples was amplified in the CJD blood test for 30 cycles. FIG. 4 shows that all three blood donor false-positives have peaks in the range of 84° C. and 86° C. The positive control had an additional peak at 91° C.

The results in Example 2 demonstrate that all CJD samples could be confirmed in the higher temperature range of between 89° C. and 92.5° C. Further, as shown in this example, the false-positive samples that were kept at RT for >4.5 hours before processing exhibited their peak reactivity between 84° C. and 86° C., but do not show the peak between 89° C. and 92.5° C. Based on these data, 30 cycles of amplification would be the preferred cycle number. In this example, samples with significant reactivity (Z value greater than 3) between 89° C. and 92.5° C. after 30 cycles should be considered as reactive.

### Example 4

### Use of Alternative Primer Sets to Identify Humans at Risk for CJD

Any number of primer pairs can be used in the detection methods of the invention. This example provides alternative exemplary primer pairs that are unrelated to the primer pair used for PCR in Examples 1-3. The primer pairs employed in the example were CJ_{—}1F and CJ_{—}1R; CJ_{—}3F and CJ_{—}3R (as in Table 2), and CJ_{—}5F and CJ_{—}5R. These primers were designed based on the three sequences in Table 2. CJ_{—}1F and CJ_{—}1R were designed based on Sequence 1; CJ_{—}3F and CJ_{—}3R and CJ_{—}5F and CJ-5R were designed based on Sequence 3.

Primer pair: CJ_1F and CJ_1R.

| | | |
|---|---|---|
| CJ_1F: | 5′-AGCCTGGGTGACAGAATGAC-3′ | (SEQ ID NO:3) |
| CJ_1R: | 5′-TCAGGGCTTATTTCCAGGAG-3′ | (SEQ ID NO:4) |

Primer pair: CJ_3F and CJ_3R.

| | | |
|---|---|---|
| CJ_3F: | 5′-GCATTGTGAACTCTCAAGCTTTATT-3′ | (SEQ ID NO:5) |
| CJ_3R: | 5′-CCTGGGTGACAGAATGAGAC-3′ | (SEQ ID NO:6) |

Primer pair: CJ_5F and CJ_5R.

| | | |
|---|---|---|
| CJ_5F: | 5′-TCTCAAGCTTTATTTTCTAATCTGA-3′ | (SEQ ID NO:7) |
| CJ_5R: | 5′-AACTAATATATAGTGAAGATTGACG-3′ | (SEQ ID NO:8) |

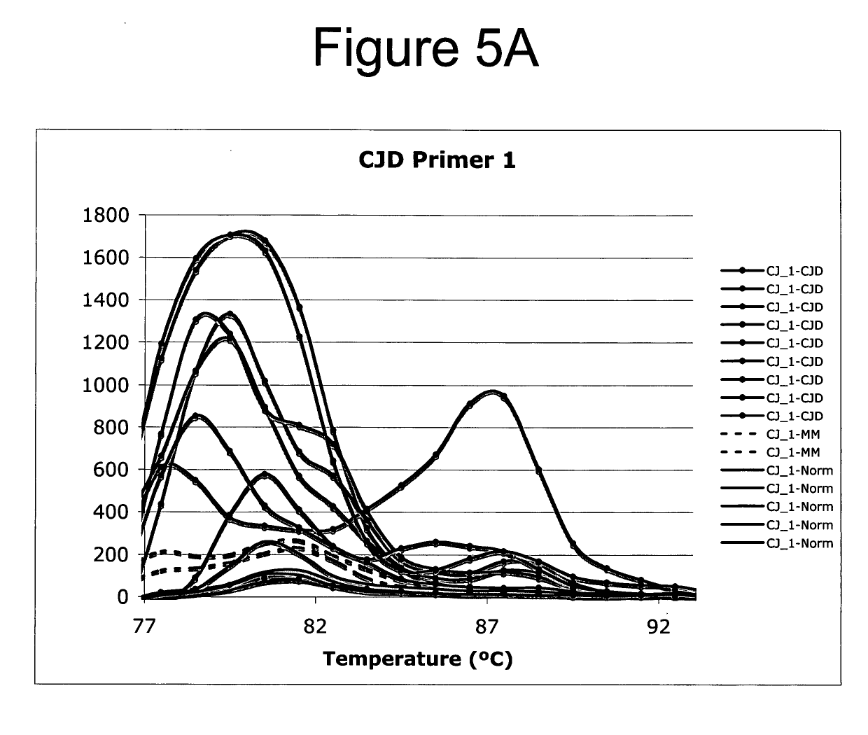

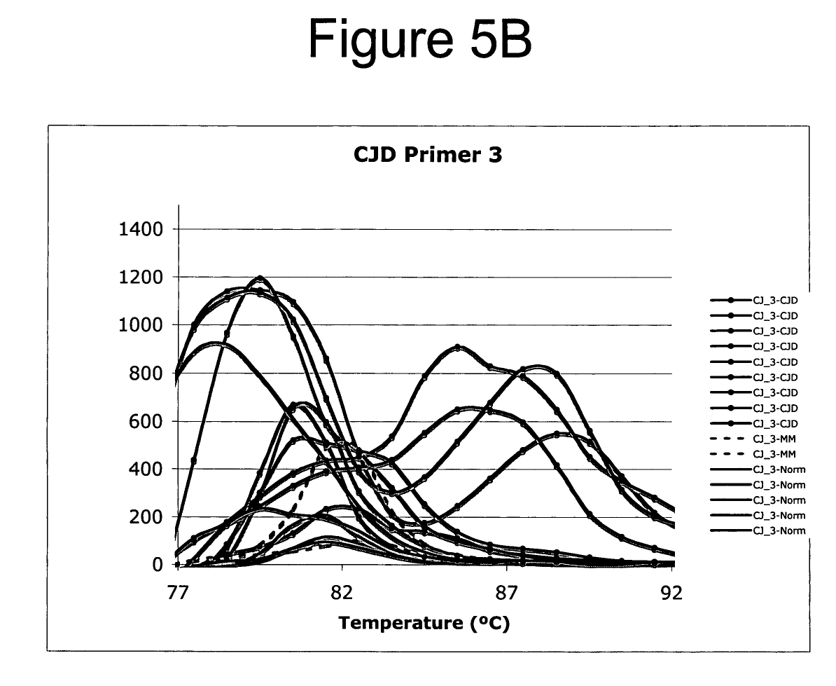

Melting curve analyses were performed on PCR analyses conducted with patient samples and samples from healthy control blood donors for each of the two exemplary primer pairs. PCR conditions were those described in Example 1 with 36 cycles. In each analysis, the melting patterns of diseased sera showed a range of similar reaction pattern, whereas a non-reactive pattern was observed in the healthy control samples. The results using Primer pairs CJ_{—}1F and CJ_{—}1R, and CJ_{—}3F and CJ_{—}3R are shown in FIGS. 5A and 5B, respectively.

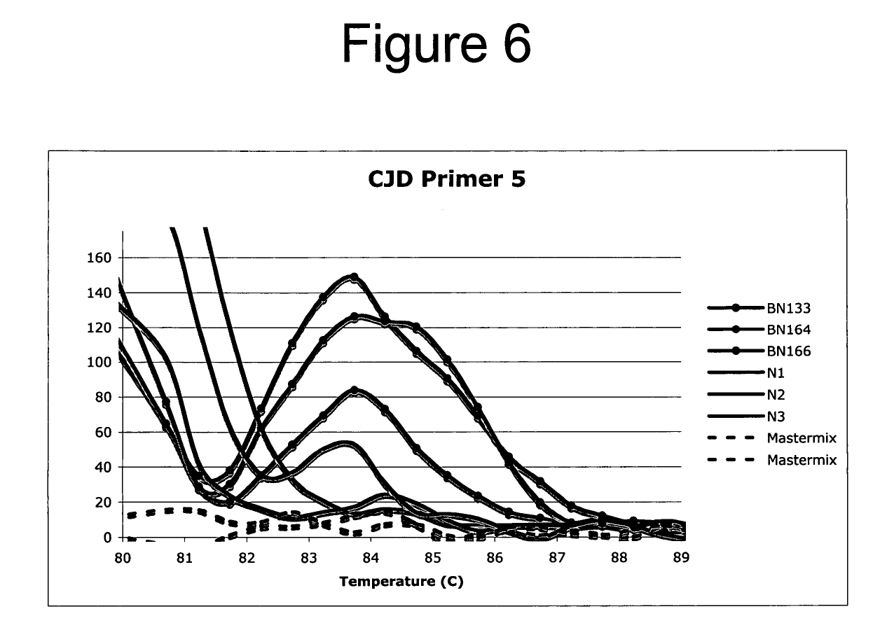

Primer pair 5 also showed separation between CJD and healthy controls (FIG. 6). Primer pair 5 was selected from the non-SINE region of Sequence 3 (Table 2) in which neither primer is from an Alu or SINE element sequence.

The above examples are provided to illustrate the invention but not to limit its scope. Other variants of the invention will be readily apparent to one of ordinary skill in the art and are encompassed by the appended claims.

All publications, patents, and patent applications cited herein are hereby incorporated by reference for all purposes.

What is claimed is:

1. A method of detecting an individual at increased risk for CJD, the method comprising:
incubating nucleic acids extracted from an acellular sample obtained from the individual with amplification primers in a test amplification reaction;
detecting reactivity of the amplification reaction that is greater than a reference amplification reaction, wherein greater reactivity is indicative of an increased risk for CJD.

2. The method of claim 1, wherein the acellular sample is serum.

3. The method of claim 1, wherein the acellular sample is plasma.

4. The method of claim 1, wherein one of the primers comprises sequences that hybridize to non-coding sequences in the human genome.

5. The method of claim 4, wherein a target sequence amplified by the primers comprises repetitive sequences.

6. The method of claim 5, wherein the repetitive sequences are SINE sequences.

7. The method of claim 6, wherein the SINE sequence are Alu sequences.

8. The method of claim 1, wherein the nucleic acid sample comprises DNA.

9. The method of claim 1, wherein the primers hybridize to the same sequences as the primers CHX-CJ-2F (SEQ ID NO:1) and CHX-CJ-2R (SEQ ID NO:2).

10. The method of claim 9, wherein at least one of the primers comprises at least 10 contiguous nucleotide of CHX-CJ-2F (SEQ ID NO:1) or CHX-CJ-2R (SEQ ID NO:2).

11. The method of claim 1, wherein the primers are CHX-CJ-2F (SEQ ID NO:1) and CHX-CJ-2R (SEQ ID NO:2).

12. The method of claim 1, wherein the primers hybridize to the same sequences as the primers CJ_{—}1F (SEQ ID NO:3) and CJ_{—}1R (SEQ ID NO:4).

13. The method of claim 1, wherein the primers are CJ_{—}1F (SEQ ID NO:3) and CJ_{—}1R (SEQ ID NO:4).

14. The method of claim 1, wherein the primers hybridize to the same sequences as the primers CJ_{—}3F (SEQ ID NO:5) and CJ_{—}3R (SEQ ID NO:6).

15. The method of claim 1, wherein the primers are CJ_{—}3F (SEQ ID NO:5) and CJ_{—}3R (SEQ ID NO:6).

16. The method of claim 1, wherein the primers hybridize to the same sequences as the primers CJ_{—}5F (SEQ ID NO:7) and CJ_{—}5R (SEQ ID NO:8).

17. The method of claim 1, wherein the primers are CJ_{—}5F (SEQ ID NO:7) and CJ_{—}5R (SEQ ID NO:8).

18. The method of claim 1, wherein the primers hybridize to SEQ ID NO:9, SEQ ID NO:10, or SEQ ID NO:11, or the complement thereof.

19. The method of claim 1, wherein the amplification characteristic is a melting curve.

20. The method of claim 1, wherein the amplification characteristic is a pattern detected by electrophoresis.

21. The method of claim 1, wherein the amplification reactions comprise a compound that specifically binds to double-stranded DNA.

22. The method of claim 21, wherein the compound is a fluorescent dye.

23. A kit comprising primers that hybridize to SEQ ID NO:9, SEQ ID NO:10, or SEQ ID NO:11.

24. A kit of claim 23, wherein the kit comprise primers that hybridize to the same sequences as the primers CHX-CJ-2F (SEQ ID NO:1) or CHX-CJ-2R (SEQ ID NO:2).

25. The kit of claim 24, wherein at least one of the primers comprises at least 10 contiguous nucleotide of CHX-CJ-2F (SEQ ID NO:1) or CHX-CJ-2R (SEQ ID NO:2).

26. The kit of claim 24, wherein the kit comprises the primers CHX-CJ-2F (SEQ ID NO:1) and CHX-CJ-2R (SEQ ID NO:2)

27. The kit of claim 23, wherein the kit comprise primers that hybridize to the same sequences as primers CJ_{—}1F (SEQ ID NO:3) and CJ_{—}1R (SEQ ID NO:4).

28. The kit of claim 23, wherein the kit comprises primers CJ_{—}1F (SEQ ID NO:3) and CJ_{—}1R (SEQ ID NO:4).

29. The kit of claim 23, wherein the kit comprises primers that hybridize to the same sequences as primers CJ_{—}3F (SEQ ID NO:5) and CJ_{—}3R (SEQ ID NO:6).

30. The kit of claim 23, wherein the kit comprises primers CJ_{—}3F (SEQ ID NO:5) and CJ_{—}3R (SEQ ID NO:6).

31. The kit of claim 23, wherein the kit comprises primers that hybridize to the same sequences as primers CJ_{—}5F (SEQ ID NO:7) and CJ_{—}5R (SEQ ID NO:8).

32. The kit of claim 23, wherein the kit comprises primers CJ_{—}5F (SEQ ID NO:7) and CJ_{—}5R (SEQ ID NO:8).

33. A kit of claim 23, further comprising a reference sample.

34. A primer having the sequence set forth in SEQ ID NO:1, SEQ ID NO:2, SEQ ID NO:3, SEQ ID NO:4, SEQ ID NO:5, SEQ ID NO:6, SEQ ID NO:7, or SEQ ID NO:8.
